(19) United States
(12) Patent Application Publication
(10) Pub. No.: US 2007/0141002 A1
(43) Pub. Date: Jun. 21, 2007

(54) Compositions and methods for imparting vibrancy

(76) Inventors: David Lee Montezinos, West Chester, OH (US); Dea Michelle Pastwa, Cincinnati, OH (US); Matthew Gus Stophlet, Williamsburg, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/580,256

(22) Filed: Oct. 12, 2006

Related U.S. Application Data
(60) Provisional application No. 60/739,676, filed on Nov. 23, 2005.
(60) Provisional application No. 60/739,677, filed on Nov. 23, 2005.

(51) Int. Cl. A61K 8/73

(52) U.S. Cl. 424/63; 424/70.13

(57) ABSTRACT

Leave-on hair care composition, comprising from about 0.1% to about 20% of a shine enhancing system comprising a first non-soluble particle reflecting a first color, a second non-soluble particle reflecting a second color, and a third non-soluble particle reflecting a third color; from about 0.001% to about 5% of a film-forming agent; and a dermatologically-acceptable carrier.

### CROSS REFERENCE TO RELATED APPLICATION(S)

This application claims the benefit of U.S. Provisional Application No. 60/739,676, filed Nov. 23, 2005; and U.S. Provisional Application No. 60/739,677, filed Nov. 23, 2005.

### FIELD OF THE INVENTION

The present invention is directed to compositions and methods for imparting vibrancy to mammalian hair.

### BACKGROUND OF THE INVENTION

The interactions of light and hair are generally dominated by reflections at the outer and inner surfaces of hair shafts. The outer surface of a hair shaft contains flat, plate-like cells called cuticle cells. The cuticle cells are arranged in an overlapping manner to yield a shingled effect, which in healthy virgin hair exists in a fairly ordered manner. This pattern generates diffraction and interference effects that can produce bright prismatic colors. The array of colors appears as distinct color spots that appear to “turn on” when natural light interacts with the hair. Under close examination (either with the naked eye or with the aid of magnifying instrumentation), the colors can be detected and can generate a shimmer. At a distance, the colors re-mix to form an overall bright whitish reflection. Healthy virgin hair accordingly exhibits a bright, prismatic shine when illuminated.

Hair can appear dull or unhealthy when the natural microstructure created by the overlapped cuticle cells is damaged or masked. Damage can arise from a variety of sources including, for example, by combing and other physical abrasion, and by chemical treatments, such as bleaching and perming. The natural microstructure may also lose some of its light interference properties if it is partially covered by sebum or, for example, materials applied to the hair via shampoo, conditioning and/or styling products. Undesirable dulling also may occur over time with hair that has been colored with dyes and other conventional penetrating colorants. Consumers may semi-permanently color hair once every few weeks or months, and in between treatments the color intensity or saturation may fade, causing the hair to appear dull. Thus, there is a need for hair care compositions to impart vibrancy to hair, such that the hair has the appearance of healthy virgin hair and/or freshly colored hair.

One way to regulate the interaction of light, and thus improve the appearance of keratinous tissues, is through the use of particulate materials. A variety of particulate materials, including interference pigments, have been used in personal care compositions, for example, in cosmetic and skin care compositions to mask imperfections or to create a more even skin tone. Applying particulate materials to hair, however, presents some difficulties. Hair may comprise one or more of a wide variety of colors, ranging from platinum blond to jet black. Thus, the underlying substrate to which the composition is applied is more varied, and choosing an appropriate color or combination of colors to achieve a desired effect is more difficult. In addition, without intending to be limited by theory, when compositions comprising particulate materials are applied to skin, the particulates may agglomerate, or “stack.” Whereas this need not be problematic for skin, and indeed, may aid in providing the desired “masking” benefit, agglomeration of particulates actually may dull the appearance of hair. Due to the differences in the structure of hair and skin, as described above, it is desirable for the particulates to form a single layer and be properly aligned with respect to the hair shaft. This allows for an interaction with light that imparts vibrancy, in particular when associated with movement. There exists a further need, therefore, to provide a hair care composition comprising a combination of particulate materials that impart vibrancy to hair.

### SUMMARY OF THE INVENTION

The present invention meets the aforementioned needs by providing leave-on hair care compositions comprising a shine enhancing system and dermatologically-acceptable carrier comprising a film-forming agent. The shine enhancing system comprises at least three non-soluble particles. Applicants believe that a composition comprising at least three shine enhancing systems can provide natural looking shine and color enhancement when applied to hair. For example, by blending non-soluble colorants (e.g., reflective pigments) having colors ranging widely across the visible spectrum, perhaps including greens and even blues, bright but unexpectedly natural looking shine can be achieved, while dullness, brassiness and fading can be diminished. Without being limited by theory, applicants believe that the mirror-like quality of these types of colorants, which adhere to the hair oriented across a broad range of angles, generates a shimmering quality that mimics the way light shines on healthy virgin hair. Furthermore, the use of multiple distinguishable hues in the choice of pigment colors enhances the visibility of this natural shine effect. When the range of colors is broad enough, a prismatic effect is achieved in which the various colors are visible upon close examination, yet blend to a bright whitish shine at a distance. This mimics the effect of sunlight on healthy virgin hair and is therefore highly desirable. Applicants further believe that vibrant hair is complex or multi-dimensional due to the plurality of colors and/or resulting contrasts, and therefore, applying a composition to hair that contains only one or two colorants (soluble or insoluble) will not necessarily impart vibrancy.

The following describe some non-limiting embodiments of the present invention.

According to the first embodiment of the present invention, a leave-on hair care composition is provided, comprising from about 0.1% to about 10% of a shine enhancing system comprising a first non-soluble particle reflecting a first color, a second non-soluble particle reflecting a second color, and a third non-soluble particle reflecting a third color; from about 0.001% to about 5% of a film-forming agent; and a dermatologically acceptable carrier.

According to a second embodiment of the present invention, a method of imparting vibrancy to mammalian hair is provided, comprising the step of applying to the hair a leave-on composition according to the first embodiment.

According to yet another embodiment of the present invention, a kit is provided, comprising at least one composition according to the first embodiment, at least one additional hair care composition, and instructions for the use of the compositions to comply with a hair treatment regimen.

### DETAILED DESCRIPTION OF THE INVENTION

While the specification concludes with claims which particularly point out and distinctly claim the invention, it is believed the present invention will be better understood from the following description. The compositions of the present invention may exist in a variety of leave-on product forms including, but not limited to, a leave-on conditioner, a styling product, mousse, gel, and combinations thereof.

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

Herein, “leave-on” means that any residual composition need not be rinsed from the hair prior to allowing the hair to dry, including the steps of applying heat, blow-drying, combing, curling or otherwise styling.

Herein, “to enhance vibrancy,” or “enhancing vibrancy” means that the compositions of the present invention impart a complex shine to a substrate (e.g., hair) such that when an observer with 20/20 vision (uncorrected or corrected) views, at a distance of 10 cm or less, a substrate treated with the compositions of the present invention and the substrate is illuminated, the observer can discern distinct shiny objects that do not appear uniform in color (i.e., hue, saturation and/or intensity) and that sparkle or shimmer when the observer changes his or her angle of observation. If the treated substrate (itself or an image of the substrate) is magnified (e.g., about 10×), different colors—variation in hue, saturation, and/or intensity—can subjectively be discerned and contrasted with respect to one another, and may be objectively identified within a color space (e.g., RGB or HSI) with image analysis tools. “Enhancing vibrancy” is understood to include one or more of the following: accentuating the color of hair, increasing (or enhancing) shine, imparting highlights (e.g., strands of hair which are lighter in color than the surrounding hair), changing the perceived color of the hair, increasing luminosity (grey scale shine), increasing saturation, and inducing a shift in hue, contrast, and/or shimmer (i.e., the blinking on and off of component color spots as the light, hair, or observer moves).

As used herein, “dermatologically-acceptable carrier” means that the carrier is suitable for topical application of a leave-on hair care composition to keratinous tissue and is compatible with the shine enhancing systems of the present invention and any other components, and will not cause any safety or toxicity concerns.

Various color models and color spaces can be used to describe the color of an object, and would be well known to one of skill in the art. One method of measuring and describing color may be found in John C. Russ, The Imaging Processing Handbook, 4^{th} Ed., CRC Press, 2002. Each of the color models/spaces includes three numerical components that relate to brightness, hue, and colorfulness. One suitable color model/space for describing color is the HSI color model, wherein hue (“H”), saturation (“S”), and intensity (“I”) properties are used to describe color. HSI can be visually described as two cones arranged end-to-end with their bases in juxtaposition and apexes in opposing relation. HSI is modeled with cylindrical coordinates. An axis extends through the juxtaposed cones and relates to intensity, with the one cone apex representing white (1, 0 coordinates) and the other cone apex representing black (0, 0 coordinates). The cone radius or radial dimension from the intensity axis relates to saturation, varying from 0 to 1. When S=0, the color is grayscale. Hue is represented by the vector angle, wherein red generally falls within a hue angle range of 0° to 30° and 310° to 360°, yellow within a hue angle range of 40° to 90°, green within a hue angle range of 70° to 160°, and blue within a hue angle range of 160° to 270°.

As used herein, the phrases “different color,” “different shade” and “different shade of color” mean a perceptible variation in intensity and/or saturation at a given hue angle, and/or a variation in hue angle. Differences in color or shade are believed perceptible when the intensity or saturation is shifted by at least about 1%, or when the hue angle shifts by at least about 1°.

As used herein, “color family” means all colors within a particular hue angle range in accordance with the HSI color space. The red color family generally falls within a hue angle range of 0° to 30° and 310° to 360°. The yellow color family generally falls within a hue angle range of 40° to 90°. The green color family generally falls within a hue angle range of 70° to 160°. And the blue color family generally falls within a hue angle range of 160° to 270°.

As used herein, “shine enhancing system” means a mixture of three or more discrete non-soluble particles, where “discrete” is understood to mean individually distinct. For example, dyes and soluble colorants would not be understood to be distinct.

As used herein, “reflect” or “reflection” means the interaction of light with an object in which the light leaves the object from a side/surface that is illuminated. “Transmit” or “transmission” means the interaction of light with an object in which the light passes through the object. As used herein, unless otherwise specified, “reflect” is understood to include both reflection and transmission.

As used herein, “transmit” or “transmission” means the interaction of light with an object in which the light leaves the object from a side/surface that is spaced apart from a side surface that is illuminated.

As used herein, “non-soluble particle” and alternatively “non-soluble colorant” means a solid material that fails to dissolve, or incompletely dissolves, in the dermatologically-acceptable carrier.

As used herein, “absorbent pigment” means a colorant that derives its color from the absorption of some wavelengths of light but not others. The light seen reflected or transmitted through such a pigment has a color that is not necessarily dependent on the geometry of the interaction of the pigment with light.

As used herein, “non-absorbent pigment” means a colorant that derives its color from the geometry of the interaction of the pigment with light. It is possible for such a pigment to appear white or nearly colorless when illuminated under circumstances that do not meet the geometric criteria required for color generation.

As used herein, “reflective pigment” means a colorant that derives its appearance through reflection (rather than transmission), and typically also reflects light in a specular fashion, thus providing the appearance of shininess (also called luster, gloss, or sheen in some circumstances). These pigments may be absorbent or non-absorbent, although typically, the term reflective pigment is used to describe absorbent pigments and more specific terms (e.g., interference pigment) are used to describe pigments that are non-absorbent or both absorbent and non-absorbent.

As used herein, “goniochromatic material” means any material that is capable of reflecting or transmitting more than one color depending on the angle of observation.

As used herein, “luminescent pigment” means a pigment that absorbs light in one range of wavelengths and then emits light in a different range of wavelengths. Fluorescent pigments and phosphorescent pigments are examples of luminescent pigments.

Measurement Methods

A variety of image analysis methods may be used to evaluate substrates treated with compositions of the present invention. Image analysis typically involves an image collection step, followed by one or more analysis steps. One image analysis method for evaluating hair switches treated with compositions of the present invention comprises the following steps. Modify a Flex Products, Inc., Direct Viewer Goniometer by replacing the peephole viewer with a mounted digital camera, and replacing the fiber optic lens assembly on the illumination swing arm with a mount for a beam probe (i.e., a fiber optic focusing lens assembly). Use a xenon arc lamp equipped with filters to cut off light outside the range of 400-700 nanometers as the light source. All the components of the light source are available from Newport Stratford, Inc. Connect the lamp to the beam probe lens assembly with a liquid light guide. Focus the light onto the hair from a distance of about 32 cm. Affix a neutral density film (25% transmission) over the exit aperture of the beam probe to reduce the intensity of the light so that good quality images can be obtained by the camera. Mount a digital camera on the modified viewer stand (e.g., a Canon EOS-1 Ds Mark II equipped with a 100 mm macro lens). Mount a 10 gram hair switch vertically under a metal plate so that the sample is visible through a 40 mm diameter hole in the middle and the hair lies flat under the plate with root ends at the top. Fix the sample holder at a 20° inclination to the angle of the camera (this is the view angle). Connect the digital camera firewire cable to a computer (Microsoft XP platform) running EOS Viewer Utility software. Set the exposures on manual with a shutter speed of 1/160, an aperture value of 4.0 and an ISO speed of 400. Set the light source at an illumination angle of 25° for the first image capture, then increment by 1° for each succeeding image capture until it reaches 65° (41 images total). Import images into the Viewer software using the EOS Capture module.

The image analysis step can be done with any of several available software packages. Photoshop (e.g., version 6.0 or version CS) with Fovea Pro 4.0 plug-ins (Reindeer Graphics) is an exemplary software package suitable for image analysis. A number of optical parameters can be analyzed from the collected images. One such parameter is integrated grey value (IGV). IGV is a calculation that can be found in the standard measurement tools associated with the program Optimas. To perform the calculation, sample all the pixels in a measurement area, multiply each grey level (intensity value) by the number of pixels at that level, and then sum the products. In Photoshop with Fovea Pro, select the same size sampling area in each image and use the IP Measure Global>Histogram plug-in to obtain a text file containing a column of pixel counts for each intensity bin. Using Microsoft Excel, open these text files, multiply and sum the intensity values, copy the calculated IGV values to one location and plot them to obtain curves of relative shininess across the range of angles imaged for each sample.

Comparisons of the IGV values of treated and untreated hair switches can be made. Increases in IGV values, in comparison to a control, can be any increase that is statistically significant at the 90% confidence level. In preferred embodiments, a treated hair switch has an IGV value that is 10% or higher than that of an untreated control.

The colorful prismatic effects that can occur when either healthy virgin hair or hair treated with compositions of the present invention illuminated by sunlight (or simulated sunlight) are visible close up (e.g, within 10 cm) and as small points (spots, dots, pixels). To measure these colors upon their appearance and disappearance as light moves across the hair, one can measure these changes on a point-by-point basis. To do this, the same single line of pixels on each image in the group of 41 can be evaluated using the IP Measure Global>Profiles plug-in in Photoshop with Fovea Pro. That tool can generate a text file for each measurement that contains RGB values for each pixel on the line at a given illumination angle. In Microsoft Excel, these RGB values can be converted to L*a*b* values using standard conversion protocols. The differences between the L*a*b* values for the same pixels on consecutive images can be used to obtain ΔL, Δa and Δb values, and these in turn used to calculate ΔE (the square root of the sum of the squares of these three values). The sum of the ΔE values for all the pixels in the line is a cumulative value for that pair of illumination angles, and a plot of the cumulative values for all the consecutive pairs in a set of images (40 values) is a plot of the color changes that occur as light sweeps across the hair. Curve fitting (e.g., via standard 3rd order polynomial trendlines available in Excel, or via Gaussian fitting tools) can indicate differences in ΔE trends between samples. Comparisons between control and treated hair switches preferably demonstrate a ΔE of at least 1 for each pixel comparison—the cumulative ΔE would accordingly be greater than or equal to the number of measured pixels.

Shine Enhancing System

The compositions of the present invention comprise from about 0.1% to about 10%, alternatively from about 0.5% to about 8%, alternatively from about 0.1% to about 5%, and alternatively from about 0.1% to about 2% of a shine enhancing system comprising one or more non-soluble particulates. The non-soluble particulate may have a size of from about 1 micron to about 150 microns, alternatively from about 1 micron to about 100 microns, alternatively from about 1 micron to about 50 microns, and alternatively from about 5 microns to about 25 microns. In one embodiment, the shine enhancing system is substantially free from dyes, where substantially free is understood to include less than 0.001%.

The non-soluble particulate may reflect a single color of visible light, herein as a “single color, non-soluble colorants,” or may be a goniochromatic colorant. The single color may fall within the red, yellow (understood to include gold), blue or green color families, as defined herein.

Exemplary single color, non-soluble colorants also include coated particles comprising one or more core materials at least partially coated with one or more coating materials. The particle may be made of one or more materials. Suitable core materials include, but are not limited to natural mica, synthetic mica, graphite, metal oxides, talc, kaolin, alumina flake, bismuth oxychloride, silica flake, ceramics, glasses, silicates (e.g., aluminosilicates and borosilicates), titanium dioxide, CaSO₄, CaCO₃, BaSO₄, and mixtures thereof. The substrate may be solid or hollow, natural or synthetic, organic or mineral. In one embodiment, the substrate is mica, iron oxide, titanium dioxide, boron nitride, interference pigments, or mixtures thereof. Suitable coating materials include, but are not limited to, metals and metallic compounds. Suitable metals include, but are not limited to, Ag, Al, Au, Cr, Cu, Ge, Mg, Mo, Ni, Rb, Se, Te, Ti, Va, W, Zn, and alloys thereof. Suitable metallic compounds, includes TiO₂, Fe₂O₃, SnO₂, Cr₂O₃, ZnO, ZnS, SnO, ZrO₂, CaF₂, Al₂O₃, BiOCL, MgF₂, CrF₃, ZnSe, SiO₂, MgO, SeO₃, SiO, ZrO₂, CeO₂, Nb₂O₅, Ta₂O₅, MoS₂, and mixtures thereof. The substrates may contain a single coating or multiple coatings.

Single color, non-soluble colorants may include other multilayered structures, with the individual layers preferably having different refractive indices. The layers may be polymeric or metallic. Multilayered films can be formed and the films separated into discrete elements or particles. Such particles are disclosed in PCT Application No. WO 99/36477 and U.S. Pat. Nos. 6,299,979 and 6,387,498. Reflective particles comprising at least two layers of polymers are sold by 3M under the tradename MirrorGlitter™. By way of example only, the layers can be made from the following polymers: polyethylene naphthalate (PEN), and its isomers, for example 2,6-, 1,4-, 1,5-, 2,7-, and 2,3-PEN, polyalkylene terephthalates, polyimides, polyetherimides, atacic polystyrenes, polycarbonates, polyalkyl methacrylates and polyalkyl acrylates, syndiotactic polystyrene, syndiotactic poly-apha-methylstyrenes, syndiotactic polydichlorostyrene, copolymers and blends of these polystyrenes, cellulose derivatives, polyalkylene polymers, fluoropolymers, chloropolymers, polysulfones, polyethersulfones, polyacrylonitriles, polayamides, silicone resins, epoxy resins, polyvinyl acetate, poehteramides, ionomeric resins, elastomers, polyurethanes, and copolymers of the like.

Single-color, non-soluble colorants also include luminescent pigments, such as for example, fluorescent pigments and phosphorescent pigments. Other single color, non-soluble colorants that are known in the art may be used in the present invention.

Suitable goniochromatic materials include multilayered interference structures, liquid-crystal coloring agents and/or photonic crystals. The goniochromatic materials comprise one or more core materials and two or more layers of a coating material, both described above. The coating material may be the same for each layer or be different.

One example of a goniochromatic material is an interference pigment. Herein, “interference pigment” means one type of interference pigment having a characteristic reflected color. For the purposes of the present specification, interference pigments are defined as particles having two or more layers of controlled thickness with different refractive indices. The interference pigments yield a characteristic reflected color from the interference of typically two, but occasionally more, light reflections, from different layers of the particle, which may be thin and plate-like. Non-limiting examples of suitable interference pigments for the composition of the present invention comprise a base substrate particle comprised of natural or synthetic mica, borosilicate glass, silica, and mixtures thereof, layered with films of TiO₂, silica, tin oxide, iron oxide, and mixtures thereof, wherein the thickness of the layers is from about 50 nm to about 300 nm.

Useful intereference pigments are available commercially from a wide variety of suppliers, for example, Rona (Timiron™), Sensient (Covapearl™), Englehard (Flamenco™), Kobo (KTZ Interval™ and Interfine™) and Eckart (Prestige™). In one non-limiting embodiment, the interference pigments have an average diameter of individual particles in the longest direction of from about 5 microns to about 75 microns, alternatively from about 5 microns to about 25 microns, and alternatively from about 10 to about 20 microns.

Goniochromatic materials with a multilayer structure comprising alternating polymer layers, for example of the type such as polyethylene naphthalate and polyethylene terephthalate, may also be used. Such coloring agents are described in PCT Application Nos. WO-A-96/19347 and WO-A-99/36478.

Pigments with a polymeric multilayer structure that may be used include those sold by 3M under the name Color Glitter. The liquid-crystal coloring agents comprise, for example, silicones or cellulose ethers onto which are grafted mesomorphic groups. Examples of liquid-crystal goniochromatic particles that may be used include, for example, those sold by Chenix and also the products sold under the name Helicone® HC by Wacker. Interference fibers with a multilayer structure may be used as the shine enhancing system. Examples of such fibers are described in European Patent Application Nos. 921,217 and 686,858, and U.S. Pat. No. 5,472,798. The multilayer structure may comprise at least two layers, each layer, which may or may not be independent of the other layer(s), being made of at least one synthetic polymer. The polymers present in the fibers may have a refractive index ranging from 1.30 to 1.82 and better still ranging from 1.35 to 1.75. The polymers that are preferred for making the fibers are polyesters such as polyethylene terephthalate, polyethylene naphthalate, polycarbonate; acrylic polymers such as polymethyl methacrylate; polyamides. Goniochromatic fibers with a polyethylene terephthalate/nylon-6 two-layer structure are sold by Teijin under the name Morphotex.

In one embodiment, the shine enhancing system comprises reflective pigments, interference pigments (with or without a base color), effect pigments, travel pigments, helicones, pearlescent pigments, luminescent pigments, fluorescent colorants, other multilayered structures that are considered single color, non-soluble colorants or goniochromatic materials as those terms are used herein, and mixtures of any of the foregoing.

Surfaces of the shine enhancing systems may be modified to improve stability, compatibility, delivery, and deposition, for example. One modification is to render the surface hydrophobic. Nonlimiting examples of suitable hydrophobic surface treatments include the addition of silicones, acrylate silicone copolymers, acrylate polymers, alkyl silane, isopropyl titanium triisostearate, sodium stearate, magnesium myristate, perfluoralcohol phosphate, perfluoropolymethyl isopropyl ether, lecithin, camauba wax, polyethylene, chitosan, lauroyl lysine, plant lipid extracts, and mixtures thereof.

Exemplary shine enhancing systems comprising mixtures of pigments are shown in Table 1 below. Some mixtures comprise a combination of reflective pigments, others a combination of reflective pigments and interference pigments, and yet others a combination of different types of interference pigments. Table 1 includes a column entitled “Color.” This referenced color was recorded from a subjective visual observation of the material. The colors for the reflective pigments generally fall within a red, yellow, green or blue color family, where gold is considered a member of the yellow color family. When the color listed for the interference pigments is indicated to be “white” or “pearl,” the interference pigment does not significantly reflect a color falling within one of the color families.

TABLE 1

| | | | Color |
|---|---|---|---|
| No | Shine enhancing system | Supplier | Family |
| 1 | 1% Covapearl ™ Bright 933 AS | SENSIENT | white |
| | 1.4% Cloisonne ™ Cerise Flambe | ENGELHARD | red |
| | 1.4% Cloisonne ™ Imperial Gold | ENGELHARD | yellow |
| | 0.7% Cloisonne ™ Blue | ENGELHARD | blue |
| | 0.7% Cloisonne ™ Green | ENGELHARD | green |
| 2 | 1% Covapearl ™ Bright 933 AS | SENSIENT | white |
| | 1.4% Chroma-Lite ™ Red | ENGELHARD | red |
| | 1.4% Chroma-Lite ™ Yellow | ENGELHARD | yellow |
| | 0.7% Chroma-Lite ™ Dark Blue | ENGELHARD | blue |
| | 0.7% Chroma-Lite ™ Green | ENGELHARD | green |
| 3 | 2.5% Covapearl ™ Bright 933 AS | SENSIENT | white |
| | 0.9% KTZ Xian Vistas | KOBO | gold |
| | 0.9% Cloisonne ™ Imperial Gold | ENGELHARD | yellow |
| | 0.9% Colorona ™ Bright Gold | EMD/RONA | gold |
| 4 | 1.2% Covapearl ™ Bright 933 AS | SENSIENT | white |
| | 1.2% KTZ Xian Vistas | KOBO | gold |
| | 1.2% Colorona ™ Bright Gold | EMD/RONA | gold |
| | 0.8% Chroma-Lite ™ Red | ENGELHARD | red |
| | 0.4% Chroma-Lite ™ Green | ENGELHARD | green |
| | 0.4% Chroma-Lite ™ Dark Blue | ENGELHARD | blue |
| 5 | 1% Prestige ™ Bright Silver | ECKART | white |
| | 1.25% Sicopearl ™ Fantastico Gold | BASF | gold |
| | 1.5% Sicopearl ™ Fantastico Ruby | BASF | red |
| | 0.85% Sicopearl ™ Fantastico Green | BASF | green |
| | 0.85% Sicopearl ™ Fantastico Rose | BASF | red |
| 6 | 1% Covapearl ™ Bright 933 AS | SENSIENT | white |
| | 1.4% Desert Reflections Canyon Sunset | ENGELHARD | orange |
| | 1.4% Desert Reflections Sunlit Cactus | ENGELHARD | green |
| | 0.7% Desert Reflections Painted Desert Plum | ENGELHARD | |
| | 0.7% Desert Reflections Midnight Sagebrush | ENGELHARD | green |
| 7 | 1% Covapearl ™ Bright 933 AS | SENSIENT | white |
| | 1.4% Duocrome ™ YR | ENGELHARD | yellow |
| | 1.4% Duocrome ™ YB | ENGELHARD | gold |
| | 0.7% Duocrome ™ GY | ENGELHARD | green |
| | 0.7% Duocrome ™ BV | ENGELHARD | blue |
| 8 | 2% Covapearl ™ Bright 933 AS | SENSIENT | white |
| | 2% Covapearl ™ Dore Gold 232 AS | SENSIENT | yellow |
| | 0.6% Covapearl ™ Blue 635 AS | SENSIENT | white |
| | 0.6% Covapearl ™ Fire Red 333 AS | SENSIENT | red |
| 9 | 2% KTZ Interfine ™ Gold | KOBO | pearl |
| | 2% KTZ Xian Vistas | KOBO | gold |
| | 0.6% KTZ Interfine ™ Blue | KOBO | pearl |
| | 0.6% KTZ Interfine ™ Red | KOBO | pearl |
| 10 | 2.67% Timiron ™ Silk Gold | EMD/RONA | pearl |
| | 1.27% Timiron ™ Silk Blue | EMD/RONA | white |
| | 1.27% Timiron ™ Silk Red | EMD/RONA | white |
| 11 | 2% Sicopearl ™ Fantastico Gold | BASF | gold |
| | 2% Sicopearl ™ Fantastico Ruby | BASF | red |
| | 0.6% Sicopearl ™ Fantastico Green | BASF | green |
| | 0.6% Sicopearl ™ Fantastico Rose | BASF | red |
| 12 | 2% Covapearl ™ Bright 933 AS | SENSIENT | white |
| | 1% Sicopearl ™ Fantastico Gold | BASF | gold |
| | 1% Sicopearl ™ Fantastico Ruby | BASF | red |
| | 0.6% Sicopearl ™ Fantastico Green | BASF | green |
| | 0.6% Sicopearl ™ Fantastico Rose | BASF | red |
| 13 | 1.6% Covapearl ™ Bright 933 AS | SENSIENT | white |
| | 1.2% Covapearl ™ Dore Gold 232 AS | SENSIENT | yellow |
| | 1.2% KTZ Interfine ™ Red | KOBO | pearl |
| | 0.6% KTZ Interfine ™ Blue | KOBO | pearl |
| | 0.6% Sicopearl ™ Fantastico Green | BASF | green |
| 14 | 1.2% Covapearl ™ Bright 933 AS | SENSIENT | white |
| | 1% Sicopearl ™ Fantastico Gold | BASF | gold |
| | 1% Sicopearl ™ Fantastico Ruby | BASF | red |
| | 1% KTZ Xian Vistas | KOBO | gold |
| | 0.5% KTZ Interfine ™ Blue | KOBO | pearl |
| | 0.5% KTZ Interfine ™ Red | KOBO | pearl |
| 15 | 1% Covapearl ™ Bright 933 AS | SENSIENT | white |
| | 1.25% Sicopearl ™ Fantastico Gold | BASF | gold |
| | 1.25% Sicopearl ™ Fantastico Ruby | BASF | red |
| | 0.85% Sicopearl ™ Fantastico Green | BASF | green |
| | 0.85% Sicopearl ™ Fantastico Pink | BASF | red |
| 16 | 0.6% Covapearl ™ Bright 933 AS | SENSIENT | white |
| | 0.75% Sicopearl ™ Fantastico Gold | BASF | gold |
| | 0.75% Sicopearl ™ Fantastico Ruby | BASF | red |
| | 0.51% Sicopearl ™ Fantastico Green | BASF | green |
| | 0.51% Sicopearl ™ Fantastico Rose | BASF | red |
| 17 | 0.2% Covapearl ™ Bright 933 AS | SENSIENT | white |
| | 0.25% Sicopearl ™ Fantastico Gold | BASF | gold |
| | 0.25% Sicopearl ™ Fantastico Ruby | BASF | red |
| | 0.17% Sicopearl ™ Fantastico Green | BASF | green |
| | 0.17% Sicopearl ™ Fantastico Rose | BASF | red |
| 18 | 1% Covapearl ™ Bright 933 AS | SENSIENT | white |
| | 0.5% Covapearl ™ Dore Gold 232 AS | SENSIENT | yellow |
| | 1% Sicopearl ™ Fantastico Gold | BASF | gold |
| | 0.85% Sicopearl ™ Fantastico Ruby | BASF | red |
| | 0.425% Sicopearl ™ Fantastico Green | BASF | green |
| | 0.425% Sicopearl ™ Fantastico Rose | BASF | red |
| | 0.5% KTZ Xian Vistas | KOBO | gold |
| | 0.5% Timiron ™ MP-20 Fine Gold | EMD/RONA | yellow |
| 19 | 1% Covapearl ™ Bright 933 AS | SENSIENT | white |
| | 1.0% Timiron MP-20 Fine Gold | EMD/RONA | gold |
| | 0.75% Covapearl ™ Dore Gold 232 AS | SENSIENT | gold |
| | 0.75% Sicopearl ™ Fantastico Gold | BASF | gold |
| | 0.75% KTZ Xian Vistas | KOBO | gold |
| | 0.48% Sicopearl ™ Fantastico Ruby | BASF | red |
| | 0.25% Sicopearl ™ Fantastico Green | BASF | green |
| | 0.25% Sicopearl ™ Fantastico Rose | BASF | red |
| 20 | 1% Covapearl ™ Bright 933 AS | SENSIENT | white |
| | 0.35% Sicopearl ™ Fantastico Gold | BASF | gold |
| | 0.85% Sicopearl ™ Fantastico Ruby | BASF | red |
| | 0.40% Sicopearl ™ Fantastico Green | BASF | green |
| | 0.85% Sicopearl ™ Fantastico Rose | BASF | red |
| | 0.3% Colorona ™ Bronze Fine | EMD/RONA | |
| | 0.3% Colorona ™ Sienna Fine | EMD/RONA | red |
| | 0.3% Colorona ™ Copper Fine | EMD/RONA | |
| | 0.85% Xirona Indian Summer | EMD/RONA | red |
| 21 | 1% Covapearl ™ Bright 933 AS | SENSIENT | white |
| | 1.4% Gemtone Ruby | ENGELHARD | red |
| | 1.4% Gemtone Topaz | ENGELHARD | green |
| | 0.7% Gemtone Sapphire | ENGELHARD | blue |
| | 0.7% Gemtone Jade | ENGELHARD | green |
| 22 | 1% Covapearl ™ Bright 933 AS | SENSIENT | white |
| | 1.4% Cellini Red | ENGELHARD | red |
| | 1.4% Cellini Yellow | ENGELHARD | yellow |
| | 0.7% Cellini Blue | ENGELHARD | blue |
| | 0.7% Cellini Green | ENGELHARD | green |
| 23 | 1% Timica Extra Bright | ENGELHARD | white |
| | 1.25% Sicopearl ™ Fantastico Gold | BASF | gold |
| | 1.25% Sicopearl ™ Fantastico Ruby | BASF | red |
| | 0.85% Sicopearl ™ Fantastico Green | BASF | green |
| | 0.85% Sicopearl ™ Fantastico Rose | BASF | red |
| 24 | 1% Timiron ™ Artic Silver | EMD/RONA | pearl |
| | 1.25% Sicopearl ™ Fantastico Gold | BASF | gold |
| | 1.25% Sicopearl ™ Fantastico Ruby | BASF | red |
| | 0.85% Sicopearl ™ Fantastico Green | BASF | green |
| | 0.85% Sicopearl ™ Fantastico Rose | BASF | red |
| 25 | 1% Flamenco Superpearl | ENGELHARD | |
| | 1.25% Sicopearl ™ Gold | BASF | gold |
| | 1.25% Sicopearl ™ Ruby | BASF | red |
| | 0.85% Sicopearl ™ Green | BASF | green |
| | 0.85% Sicopearl ™ Rose | BASF | red |
| 26 | 1% Covapearl ™ Bright 933 AS | SENSIENT | white |
| | 1.4% Cloisonne ™ Cerise Flambe | ENGELHARD | red |
| | 1.4% Covapearl ™ Fire Red 333 AS | SENSIENT | red |
| | 0.7% Cloisonne ™ Rouge Flambe | ENGELHARD | red |
| | 0.7% Cellini Red | ENGELHARD | red |
| 27 | 1% Covapearl ™ Bright 933 AS | SENSIENT | white |
| | 0.8% Cloisonne ™ Cerise Flambe | ENGELHARD | red |
| | 0.8% Cloisonne ™ Imperial Gold | ENGELHARD | yellow |
| | 0.4% Cloisonne ™ Blue | ENGELHARD | blue |
| | 0.4% Cloisonne ™ Green | ENGELHARD | green |
| | 0.8% Covapearl ™ Red 333 AS | SENSIENT | red |
| | 0.5% Cellini Red | ENGELHARD | red |
| | 0.5% Cloisonne ™ Rouge Flambe | ENGELHARD | red |
| 28 | 1% Covapearl ™ Bright 933 AS | SENSIENT | white |
| | 1% Timica Golden Bronze | ENGELHARD | |
| | 1.2% Cloisonne ™ Cerise Flambe | ENGELHARD | red |
| | 1.2% Covapearl ™ Fire Red 333 AS | SENSIENT | red |
| | 0.5% Cloisonne ™ Rouge Flambe | ENGELHARD | red |
| | 0.5% Cellini Red | ENGELHARD | red |
| 29 | 1% Covapearl ™ Bright 933 AS | SENSIENT | white |
| | 1% Cloisonne ™ Nu-Antique Copper | ENGELHARD | |
| | 1.2% Cloisonne ™ Cerise Flambe | ENGELHARD | red |
| | 1.2% Covapearl ™ Fire Red 333 AS | SENSIENT | red |
| | 0.5% Cloisonne ™ Rouge Flambe | ENGELHARD | red |
| | 0.5% Cellini Red | ENGELHARD | red |
| 30 | 2.5% Mearlmaid AA | ENGELHARD | pearl |
| | 0.9% KTZ Xian Vistas | KOBO | gold |
| | 0.9% Cloisonne ™ Imperial Gold | ENGELHARD | yellow |
| | 0.9% Colorona ™ Bright Gold | EMD/RONA | gold |
| 31 | 2.5% Mearlmaid BU | ENGELHARD | pearl |
| | 0.9% KTZ Xian Vistas | KOBO | gold |
| | 0.9% Cloisonne ™ Imperial Gold | ENGELHARD | yellow |
| | 0.9% Colorona ™ Bright Gold | EMD/RONA | gold |
| 32 | 1.0% Covapearl ™ Bright 933 AS | SENSIENT | white |
| | 1.4% KTZ Xian Vistas | KOBO | gold |
| | 1.4% KTZ Bolero Cromatico | KOBO | red |
| | 0.5% KTZ Foliage Flutter | KOBO | red |
| | 0.5% KTZ Winterveld | KOBO | red |
| | 0.4% KTZ Jaipur Sunset | KOBO | |
| 33 | 1.0% Covapearl ™ Bright 933 AS | SENSIENT | white |
| | 1.4% DG14 DermaGlow ™ Fire Orange | DAYGLO COLOR | red |
| | 1.4% DGY410 DermaGlow ™ Yellow 410 | DAYGLO COLOR | yellow |
| | 0.7% DGP20 Cosmetic Phosphorescent Pigment | DAYGLO COLOR | green |
| | 0.7% DG-20 DermaGlow ™ Venetian Violet | DAYGLO COLOR | blue |
| 34 | 1% Prestige ™ Soft Silver | ECKART | white |
| | 1.25% Sicopearl ™ Fantastico Gold | BASF | gold |
| | 1.25% Sicopearl ™ Fantastico Ruby | BASF | red |
| | 0.85% Sicopearl ™ Fantastico Green | BASF | green |
| | 0.85% Sicopearl ™ Fantastico Pink | BASF | red |
| 35 | 1% Prestige Silver Star | ECKART | white |
| | 1.25% Sicopearl ™ Fantastico Gold | BASF | gold |
| | 1.25% Sicopearl ™ Fantastico Ruby | BASF | red |
| | 0.85% Sicopearl ™ Fantastico Green | BASF | green |
| | 0.85% Sicopearl ™ Fantastico Pink | BASF | red |
| 36 | 1% Prestige Bright Silver Star | ECKART | white |
| | 1.25% Sicopearl ™ Fantastico Gold | BASF | gold |
| | 1.25% Sicopearl ™ Fantastico Ruby | BASF | red |
| | 0.85% Sicopearl ™ Fantastico Green | BASF | green |
| | 0.85% Sicopearl ™ Fantastico Pink | BASF | red |
| 37 | 2.6% KTZ Interfine ™ Blue | KOBO | blue |
| | 2.6% KTZ Interfine ™ Gold | KOBO | yellow |
| 38 | 2.6% KTZ Interfine ™ Blue | KOBO | blue |
| | 2.6% KTZ Interfine ™ Green | KOBO | green |
| 39 | 2.6% KTZ Interfine ™ Blue | KOBO | blue |
| | 2.6% KTZ Interfine ™ Red | KOBO | red |
| 40 | 2.6% KTZ Interfine ™ Gold | KOBO | yellow |
| | 2.6% KTZ Interfine ™ Green | KOBO | green |
| 41 | 2.6% KTZ Interfine ™ Gold | KOBO | yellow |
| | 2.6% KTZ Interfine ™ Red | KOBO | red |
| 42 | 2.6% KTZ Interfine ™ Green | KOBO | green |
| | 2.6% KTZ Interfine ™ Red | KOBO | red |
| 43 | 1.7% Colorona ™ Blackstar Gold | EMD/RONA | gold |
| | 1.7% Colorona ™ Blackstar Red | EMD/RONA | red |
| | 0.9% Colorona ™ Blackstar Blue | EMD/RONA | blue |
| | 0.9% Colorona ™ Blackstar Green | EMD/RONA | green |
| 44 | 1.74% Colorona ™ Bronze Fine | EMD/RONA | |
| | 1.74% Colorona ™ Copper Fine | EMD/RONA | |
| | 1.74% Colorona ™ Sienna Fine | EMD/RONA | |
| 45 | 1.74% Colorona ™ Bright Gold | EMD/RONA | gold |
| | 1.74% Colorona ™ Red Gold | EMD/RONA | gold |
| | 1.74% Colorona ™ Dore Gold | EMD/RONA | yellow |
| 46 | 1.74% Timica ™ Golden Bronze | ENGELHARD | |
| | 1.74% Timica ™ Nu-Antique Bronze | ENGELHARD | |
| | 1.74% Timica ™ Nu-Antique Copper | ENGELHARD | |
| 47 | 1.74% Cloisonne ™ ™ Sparkle Bronze | ENGELHARD | |
| | 1.74% Cloisonne ™ ™ Sparkle Copper | ENGELHARD | |
| | 1.74% Duocrome ™ Sparkle BR | ENGELHARD | |
| 48 | 1.74% Lowa ™ Color Variable Pearls Peony OG73 | ARYSTA | red |
| | 1.74% Lowa ™ Starlight Sparkle Orange | ARYSTA | red |
| | 1.74% Lowa ™ Starlight Sparkle Golden | ARYSTA | gold |
| 49 | 1.0% Synmica ™ Super | SUN CHEMICAL | white |
| | 1.2% Sunshine Glitter Khaki Gold | SUN CHEMICAL | gold |
| | 1.2% Sunchroma ™ D&C Red 7 Ca Lake | SUN CHEMICAL | red |
| | 1.0% Sunshine Super Deep Blue | SUN CHEMICAL | blue |
| | 1.0% Soft-Tex ™ Brown Iron Oxide | SUN CHEMICAL | |
| 50 | 1.0% Synmica ™ Super | SUN CHEMICAL | white |
| | 1.4% C37-038 D&C Red Al Lake | SUN CHEMICAL | red |
| | 1.4% C23-009 D&C Red 36 | SUN CHEMICAL | red |
| | 1.4% Sunchroma ™ D&C Red No. 6 Ba Lake | SUN CHEMICAL | red |
| 51 | 1.0% Synmica ™ Super | SUN CHEMICAL | white |
| | 1.1% Soft-Tex ™ D&C Red 7 Ca Salt | SUN CHEMICAL | red |
| | 1.0% Sunchroma ™ D&C Red 21 Al Lake | SUN CHEMICAL | red |
| | 1.1% Sunchroma ™ D&C Red 7 Ca Lake (lot | SUN CHEMICAL | red |
| | 5A9606) | | |
| | 1.0% Sunchroma ™ D&C Red 7 Ca Lake (lot | SUN CHEMICAL | red |
| | 591133) | | |

Mixtures of pigments that reflect an array or gamut of colors, including red, yellow, green, lue may be used in hair care compositions appropriate for any base hair color consumer, such as blonde, brunette, red, etc. Applicants however, believe some examples are more suitable for application to a specific base hair color. For example, in one embodiment, the mixture is substantially free from pigments with a blue base color for use in compositions intended for application to light colored hair (e.g., blonde or light brown), because the blue may be visible against the base hair color background and may make the hair look unattractive or dirty.

Applicants further believe that a mixture of pigments having different shades of a single color family is suitable for certain base hair colors, such as, for example, blonde or red hair. Such mixtures preferably comprise an interference pigment. In this case, desirable shine is combined with extra color enhancement for red and blonde shaded hair by foregoing the prismatic effect, but maintaining shimmer and color. Applicants believe that the choice of multiple shades within a single color family is important for enhancing the visibility of this effect.

With reference again to Table 1, one embodiment suitable for red-shaded hair is mixture number 29, comprising five different reflective pigments included in the red color family, and one interference pigment. One embodiment suitable for blonde-shaded hair is mixture number 3, comprising three different reflective pigments included in the yellow color family, and one interference pigment. One embodiment suitable for dark brown and/or black shaded hair is mixture number 43, comprising a specular reflection of gold, red, blue or green, and a diffuse reflection of brown or black.

The mixtures of pigments included in Table 1 are exemplary only, and other mixtures of pigments may be employed in hair care compositions in accordance with the present invention. Dermatologically-acceptable carrier

The compositions of the present invention comprise from about 50% to about 99.9% of a dermatologically-acceptable carrier. In one embodiment, the dermatologically-acceptable carrier is an aqueous carrier and comprises a water phase which can optionally include other liquid, water-miscible or water-soluble solvents such as lower alkyl alcohols, e.g., C₁-C₅ alkyl monohydric alcohols, and alternatively C₂-C₃ alkyl alcohols.

Other suitable carriers include, but are not limited to, water, silicone oil, organic solvents such as C₁-C₆ alkanols, carbitol, acetone, and combinations thereof. Specific examples of suitable C₁-C₆ alkanols include, but are not limited to, ethanol, n-propanol, isopropanol, n-butanol, amyl alcohol, and mixtures thereof. Other suitable carriers include emulsions comprising an aqueous phase and an oil phase. The oils may be derived from animals, plants, or petroleum, may be natural or synthetic, and may include silicone oils. Emulsion carriers include, but are not limited to, oil-in-water, water-in-oil, water-in-oil-in-water, and oil-in-water-in-silicone emulsions.

In one embodiment, the composition comprises less than 5%, and alternatively less than 2%, of one or more surfactants. Alternatively, the composition is substantially free from surfactants, where “substantially free” is understood to mean less than 1% of one or more surfactants.

Film-forming agents

The compositions of the present invention comprise from about 0.001% to about 5% of a film-forming agent, useful to facilitate deposition of the shine enhancing systems to various substrates without significant agglomeration. Suitable film formers include, but are not limited to, silicone film forming polymers, siloxysilicates, polyrnethylsilsesquioxane, silicone/(meth)acrylate copolymers, silicone esters, copolymers of vinylprrolidone and long-chain-olefins, sugar amines and polymers comprising sugar amines.

In one embodiment, the film-forming agent comprises one or more sugar amines, also known as amino sugars. As used herein, “sugar amine” refers to an amine derivative of a six-carbon sugar. Examples of sugar amines that are useful herein include glucosamine, N-acetyl glucosamine, mannosamine, N-acetyl mannosamine, galactosamine, N-acetyl galactosamine, and mixtures thereof. In one embodiment, the sugar amine is N-acetyl glucosamine. In one embodiment, the composition comprises from about 0.001% to about 1%, and alternatively from about 0.01% to about 0.5%, of a sugar amine. Alternatively, the ratio of the percentage of shine-enhancing agent to the percentage of film-forming agent is about 10:1.

Optional Components

The compositions of the present invention may include a wide variety of optional components, including among them any of the types of components known in the art for use in leave-on hair care products. The optional components include, but are not limited to, silicones, gums, resins, solvents, non-solubilized particulates other than those described with reference to the shine enhancing system, surfactants, dispersing aids, deposition aids, propellants, moisturizers, and conditioning agents. Other optional components include hair-hold polymers; detersive surfactants, such as anionic, nonionic, amphoteric, and zwitterionic surfactants; thickening agents and suspending agents, such as xanthan gum, guar gum, hydroxyethyl cellulose, methyl cellulose, hydroxyethylcellulose, starch, and starch derivatives; viscosity modifiers, such as methanolamides of long chain fatty acids; crystalline suspending agents; pearlescent aids, such as ethylene glycol distearate; preservatives, such as benzyl alcohol, methyl paraben, propyl paraben, and imidazolidinyl urea; polyvinyl alcohol; ethyl alcohol; pH adjusting agents, such as citric acid, sodium citrate, succinic acid, phosphoric acid, sodium hydroxide, sodium carbonate; salts, such as potassium acetate and sodium chloride; hair oxidizing agents, such as hydrogen peroxide, perborate and persulfate salts; hair reducing agents, such as thioglycolates; perfumes; sequestering agents, such as disodium ethylenediamine tetra-acetate; polymer plasticizing agents, such as glycerin, disobutyl adipate, butyl stearate, and propylene glycol.

The composition of the present invention may comprise a styling agent, useful to facilitate styling, for example combing, drying, blow-drying, heating, curling (with or without heat), etc. Non-limiting examples of suitable styling agents includes polyalkylene glycols, polyethylene/polypropylene glycol copolymers, polyethylene/polypropylene diol copolymers, polyglycerins, polysaccharide styling polymers, polyvinylpyrrolidone (PVP), copolymers of PVP and methylmethacrylate, copolymers of PVP and vinylacetate (VA), poylvinyl alcohol (PVA), copolymers of PVA and crotonic acid, copolymers of PVA and maleic anhydride, hydroxypropyl cellulose, hydroxypropyl guar gum, sodium polystyrene sulfonate, PVP/ethymethacrylate/methacrylic acid terpolymer, vinyl acetate/crotonic acid/vinyl neodecanoate copolymer, octylacrylamide/acrylates copolymer, monoethy ester of poly(methyl vinyl ether-mateic acid), and octylacrylamide/acrylate/butylaminoethyl methacrylate copolymers,acrylic acid/t-butyl acrylate copolymers, dimethylaminoethyl methacrylate/isobutyl methacrylate/2-ethylhexyl-methacrylate terpolymers, t-butylacrylate/acrylic acid copolymers, and silicone grafted terpolymers, e.g., t-butylacrylate/ acrylic acid/PDMS, and mixtures thereof.

Nonlimiting examples of suitable polysaccharide styling polymers include anionic polysaccharides, cationic polysaccharides, and mixtures thereof. Cationic polysaccharide styling polymers include, but are not limited to, copolymers of hydroxyethylcellulose and diallyldimethyl ammonium chloride (referred to in the industry by CTFA as Polyquatemium-4) such as those commercially available from National Starch (Bridgewater, N.J.) under the CELQUAT tradename (e.g., CELQUAT L-200 and CELQUAT H-100); and cationic quaternary ammonium-containing polymers, including, for example, homopolymers of hydroxyethyl cellulose reacted with a trimethyl ammonium substituted epoxide, (referred to in the industry by CTFA as Polyquaternium-10) such as those commercially available from Amerchol Corp. (Edison, N.J.) under the UCARE tradename (e.g., UCARE POLYMER JR-400, and UCARE POLYMER LR400), and those commercially available from National Starch (Bridgewater, N.J.) under the CELQUAT tradename (e.g., CELQUAT SC 230 and CELQUAT SC 240). Nonionic cellulose derivatives, such as methyl and hydroxyalkyl celluloses may also be used. For example, hydroxyethyl celluloses sold under various tradenames (e.g., Natrosel by Aqualon and Cellosize by Union Carbide), methyl celluloses available from Dow Chemical Company, and hydroxypropyl methylcelluloses available from Aqualon.

The compositions of the present invention may also include one or more conditioning agents. Exemplary conditioning agents include cationic crosslinked polymeric conditioning agents and silicone conditioning agents, such as those described in U.S. Pat. No. 5,674,478 (including the conditioning agents disclosed in the references incorporated therein). The cationic crosslinked polymeric conditioning agents can be characterized by the general formula: (A)ₘ(B)ₙ(C)ₚ wherein (A) is a dialkylaminoalkyl acrylate monomer or its quaternary ammonium or acid addition salt, (B) is a dialkylaminoalkyl methacrylate monomer or its quaternary ammonium or acid addition salt, (C) is a nonionic monomer that is polymerizable with (A) or (B), m is an integer of 0 or greater, n is an integer of 0 or greater, and p is an integer of 0 or greater, wherein either m or n, or both, must be 1 or greater. The silicone conditioning agents may include polydimethylsiloxanes, polyalkyl siloxanes, polyaryl siloxanes, polyalkylaryl siloxanes, and polyether siloxane copolymers. Additional exemplary conditioning agents include low melting point fatty alcohols, such as, for example, unsaturated monohydric straight chain fatty alcohols, saturated branched chain fatty alcohols, saturated C₈-C₁₂ straight chain fatty alcohols, and mixtures thereof. Still other exemplary conditioning agents include, for example, cationic surfactants, cationic polymers, nonvolatile silicones (including soluble and insoluble silicones), nonvolatile hydrocarbons, saturated C₁₄ to C₂₂ straight chain fatty alcohols, nonvolatile hydrocarbon esters, and mixtures thereof.

The examples included herein may further provide optional components to the extent not described above. It is to be understood, that optional components other than those described above or included in the examples may also be employed in compositions according to the present invention.

Methods of Use

The compositions of the present invention may be applied to wet or to dry hair. In one embodiment, the composition is applied to wet hair. Additionally or alternatively, the composition may be applied to hair that has been previously treated, for example “permed” (semi-permanently curled or waved), straightened, colored, and/or subjected to other types of permanent or semi-permanent treatments. Additionally or alternatively, the composition may be applied to damaged hair, for example, hair that is dry, brittle, dull, frizzy, has split ends, and/or otherwise damaged, for example by exposure to heat, UV-radiation, environmental chemicals, pool chemicals, etc. The amount applied will vary according to factors such as the length of the hair and the amount of vibrancy, shine, etc. desired, however one example of a typical amount may comprise from about 1 g to about 10 g of composition. The composition may be applied by a variety of means, including with the palm of the hand, with fingertips, spraying, extruding directly onto the hair, or with an implement such as a comb, brush, sponge, etc. The composition further may be applied to any portion of the hair, for example to a portion of the hair nearest the scalp, to a portion of the hair furthest from the scalp, or to any portion of the hair where increased vibrancy is desired.

After application, the composition is intended to remain on the hair until the next washing. After application, the hair may be allowed to dry naturally, or styled, for example by combing, brushing, drying with cool or heated air, curling (with or without application of heat), straightening, binding or otherwise fixing, etc.

Kit

The compositions of the present invention may form part of a kit. The kit may comprise a packaging unit comprising at least one composition described herein, for example, an outer packaging unit, which in turn may comprise one or more inner packaging units. The inner and outer packaging units may be of any type suitable for containing, presenting and/or reasonably protecting from damage the contents of the kit. Alternatively, the kit may comprise an array of products sold individually to a consumer or professional for use in a salon.

The kit may comprise one or more compositions described herein, in addition to one or more additional compositions and/or components. Examples of suitable additional compositions include, but are not limited to, hair dyes and colorants, hair bleaching or lightening agents, a shampoo, a conditioner and/or styling agent. Examples of additional components include implements, applicators, combs, brushes, styling aids, decorative adornments, one or more orally ingestible dietary supplements, a delivery enhancement device (e.g., an ultrasonic and/or electrical device), instructions for use of the device, etc. The kit further may include instructions for use of the compositions of the present invention in conjunction with the additional compositions and/or components of the kit, for example as part of a hair treatment regimen. For example, the instructions may communicate use of a first hair care composition having a colorant capable of penetrating hair shafts, use of a second hair care composition having a shine enhancing agent, and communicating the frequency of using the first and second hair care compositions. Alternatively, the treatment regimen may comprise the steps of washing the hair, conditioning the hair, rinsing the hair, applying a composition comprising a shine enhancing agent, styling the hair, and combinations of any of the foregoing. Alternatively, the treatment regimen may comprise the step of applying an oxidative or other colorant to the hair, and applying a composition of the present invention to the hair, wherein the frequency of applying the composition of the present invention is greater or lesser than the frequency of applying the colorant to the hair. By way of illustration only, a consumer or professional stylist may be instructed to apply an oxidative or other colorant to the hair once every 6 months, and to apply the composition of the present invention once every month.

### EXAMPLES

### Example 1

A hair styling product suitable for use by one or more methods described herein may be prepared as follows:

| | Anticipated weight |
|---|---|
| Component | % in finished product |
| Water | Quantum sufficiens (q.s.) |
| Shine enhancing system¹ | 5.000 |
| Acrylates/Beheneth-25 Methacrylate | 3.000 |
| Copolymer (Aculyn-28) | |
| Hydroxyethylcellulose (HHR250) | 0.666 |
| Laureth-23 | 0.600 |
| Acetyl Glucosamine | 0.500 |
| Benzyl Alcohol | 0.500 |
| Aminomethyl Propanol | 0.316 |
| DMDM Hydantoin (Glydant) | 0.370 |
| Aloe | 0.250 |
| Disodium EDTA | 0.115 |
| Perfume | 0.100 |
| Niacinamide | 0.010 |
| DL Panthenol | 0.020 |
| Panthenyl Ethyl Ether | 0.090 |

¹Any of the examples of suitable shine enhancing systems described in Table 1 may be used, in addition to other shine enhancing systems described elsewhere herein.

Add water to a mixing vessel and bring it to a suitable temperature for making the hair care styling product, and begin agitation. Add Disodium EDTA and DMDM Hydantoin and continue mixing. Add Acrylates/Beheneth-25 Methacrylate Copolymer and mix until evenly dispersed. Emulsify perfume and Panthenyl Ethyl Ether in hot Laureth-23, and then add to the main batch. Add Niacinamide and Acetyl Glucosamine, and ensure solids are dispersed evenly. Add Benzyl Alcohol and DL Panthenol. Add Hydroxyethylcellulose, and mix until evenly dispersed and free of lumps. Add shine enhancing system into the process stream and mix until evenly dispersed. Add Aminomethyl Propanol to the mixing vessel and continue to mix until the batch is homogeneous. The amount of Aminomethyl Propanol added can be adjusted to control the pH at a desired level. Apply the composition to clean, wet hair. Do not rinse or remove excess composition from the hair until the next washing. Style as desired.

### Example 2

A hair conditioner suitable for use by one or more methods described herein may be prepared as follows:

| | Anticipated weight |
|---|---|
| Component | % in finished product |
| Water | q.s. |
| Shine enhancing system¹ | 2.000 |
| L-Glutamic Acid | 0.640 |
| Stearamidopropyldimethylamine | 2.300 |
| (SAPDMA) | |
| Cetyl Alcohol | 2.500 |
| Stearyl Alcohol | 4.500 |
| Dimethicone/Cyclomethicone (15/85 | 4.200 |
| Blend) | |
| Ethylene Diamine Tetraacetic Acid | 0.100 |
| (EDTA) | |
| Benzyl Alcohol | 0.400 |
| Kathon CG | 0.0005 |
| DL Pantyl | 0.050 |
| DL-Panthenol | 0.050 |

¹Any of the examples of suitable shine enhancing systems described in Table 1 may be used, in addition to other shine enhancing systems described elsewhere herein.

In a suitable mixing vessel, form a lamellar gel matrix as follows: obtain deionized water at a temperature of about 85° C., and add Stearamidopropyldimethylamine, Cetyl Alcohol, Stearyl Alcohol, and L-glutamic Acid. Maintain the mixture at a temperature of about 85° C. for 5 minutes, such that the ingredients are homogenized and no solids are observed. Cool the mixture to about 55° C., and maintain at this temperature until a lamellar gel matrix forms. Add the shine enhancing system and mix for about 15 minutes at a temperature of about 35° C. Add the remaining ingredients also to the lamellar gel matrix. Apply the composition to a desired portion of dry hair. Do not rinse or remove excess composition from the hair until the next washing. Style as desired.

### Example 3

A hair conditioner suitable for use by one or more methods described herein may be prepared as follows:

| | Anticipated weight |
|---|---|
| Component | % in finished product |
| Water | q.s. |
| Shine enhancing system¹ | 0.1000 |
| Propylene Glycol Dicaprylate Dicaprate | 1.000 |
| (Polyquaterium-37) | |
| Polyethylene Glycol (Peg-4) | 0.500 |
| Polyquaternium-39 | 0.500 |
| Dimethicone and Dimethiconol Blend | 0.500 |
| (Q2-1403) | |
| Disodium EDTA | 0.127 |
| Phenoxyethanol | 0.200 |
| Methyl Paraben | 0.200 |

¹Any of the examples of suitable shine enhancing systems described in Table 1 may be used, in addition to other shine enhancing systems described elsewhere herein.

In a suitable mixing vessel, add deionized water at room temperature and start mixing. Add Propylene Glycol Dicaprylate Dicaprate (Polyquatemium-37). Next, add the shine enhancing system. Add remaining ingredients. Apply the composition to a desired portion of wet, previously color-treated hair. Do not rinse or remove excess composition from the hair until the next washing. Style as desired.

### Example 4

A hair conditioner suitable for use by one or more methods described herein may be prepared as follows:

| | Anticipated weight |
|---|---|
| Component | % in finished product |
| Water | q.s. |
| Shine enhancing system¹ | 5.000 |
| Triethanolamine | 0.820 |
| Carboxyvinylpolymer (Carbopol 981) | 0.100 |
| Acrylates/C10-C30 Alkyl Acrylate | 0.700 |
| Crosspolymer (Pemulen TR-1) | |
| Dimethicone and Dimethiconol Blend | 0.500 |
| (Q2-1403) | |
| Polyquaternium-39 | 0.100 |
| Peg-4 | 0.100 |
| Phenoxyethanol | 0.200 |
| Methyl Paraben | 0.200 |
| Disoium EDTA | 0.127 |

¹Any of the examples of suitable shine enhancing systems described in Table 1 may be used, in addition to other shine enhancing systems described elsewhere herein.

In a suitable mixing vessel, add water and disperse the carboxyvinylpolymer and the Acrylates/C10-C30 Alkyl Acrylate Crosspolymer therein. Mix the dispersion with vigorous agitation and heat to about 50° C. Cool the mixture below 40° C., and then add Triethanolamine. Neutralize and then add the remaining ingredients. Apply the composition to wet, previously permed, hair. Do not rinse or remove excess composition from the hair until the next washing. Style as desired.

### Example 5

A hair care mousse formula suitable for use by one or more methods described herein may be prepared as follows:

| Component | Anticipated weight % in finished product |
|---|---|
| Water | q.s. |
| Shine enhancing system¹ | 10.000 |
| Polyquaternium-4 | 2.750 |
| Pareth-91-8 | 0.229 |
| Propylene Glycol | 0.458 |
| DMDM Hydantoin | 0.356 |
| Citric Acid (50% Solution) | 0.0183 |
| Disodium EDTA | 0.0940 |
| Propellant A-55 | 6.0000 |

¹Any of the examples of suitable shine enhancing systems described in Table 1 may be used, in addition to other shine enhancing systems described elsewhere herein.

Add water to a mixing vessel and bring to a suitable temperature (26° C. to 85° C.) for the incorporation of the Polyquaternium-4. Add the Polyquatemium-4 into the process stream in the mixing vessel allowing for the material to be evenly dispersed. Add the Disodium EDTA and a portion of the Citric Acid to the mixing vessel, mix until the solids are incorporated. Once the solution is apparently clear of solids, set the vessel temperature to new process conditions (26° C. to 38° C.), and then add the Pareth 91-8 and the Propylene Glycol. Add the DMDM Hydantoin once the new process conditions are met. Incorporate the shine enhancing system into the process stream. Mix until evenly distributed. Adjust the pH with additional Citric Acid as desired. Fill an aerosol container to the appropriate level with the above made concentrate, pull a vacuum and crimp a valve in place. Add required propellant blend through the valve, not to exceed 6% of volume. Apply the composition to clean, wet hair. Do not rinse or remove excess composition from the hair until the next washing. Style as desired.

### Example 6

A clear conditioning formula suitable for use by one or more methods described herein may be prepared as follows:

| Water | q.s. |
|---|---|
| Shine enhancing system^{1,2,3,4,5} | As indicated^{1,2,3,4,5} |
| Polyquaternium 37 | 1.50 |
| Solubilisant ™ LRI⁶ | 0.30 |
| Methylparaben | 0.20 |
| Propylparaben | 0.20 |
| Propylene Glycol | 40.00 |
| Genamin ™ KDMP⁷ | 0.80 |
| Hydrogenated Castor Oil PEG-40 | 0.30 |
| Perfume Pearl | 0.20 |

¹The following shine enhancing system is particularly suitable for use on brunette hair: 1.00% Covapearl ™ Bright (SENSIENT), 1.40% Chroma Lite ™ Red (ENGLEHARD), 1.40% Chroma Lite ™ Yellow (ENGLEHARD), 0.7% Chroma Lite ™ Dark Blue (ENGLEHARD), 0.70% Chroma Light ™ Green (ENGLEHARD).
²The following shine enhancing system is particularly suitable to create red highlights on brunette hair: 1.00% Covapearl ™ Bright (SENSIENT), 1.40% Cloisonne ™ Cerise Flambe (ENGLEHARD), 1.40% Cloisonne ™ Imperial Gold (ENGLEHARD), 0.7% Cloisonne ™ Green (ENGLEHARD), 0.70% Chroma Light ™ Dark Blue (ENGLEHARD).
³The following shine enhancer is particularly suitable to create golden-brown highlights on brunette hair: 1.00% Covapearl ™ Bright (SENSIENT), 1.40% Desert Reflection Canyon Sunset (ENGLEHARD), 1.40% Desert Reflection Sunlit Cactus (ENGLEHARD), 0.7% Desert Reflection Painted Desert Plum (ENGLEHARD), 0.70% Desert Reflection Midnight Sagebrush (ENGLEHARD).
⁴The following shine enhancer is particularly suitable for enhancing the vibrancy of blonde hair: 0.6% KTZ Interfine ™ Blue (KOBO), 2.00% KTZ Interfine ™ Gold (KOBO), 0.60% KTZ Interfine ™ Red (KOBO), 2.00% Xian Vistas (KOBO).
⁵The following shine enhancing system is particularly suitable for enhancing vibrancy of red hair: 1.00% Covapearl ™ Bright (SENSIENT), 1.00% Cloisonne ™ Nu-Antique Copper (ENGELHARD), 1.20% Cloisonne ™ Cerise Flambe (ENGELHARD), 1.20% Covapearl ™ Fire Red (SENSIENT), 0.50% Cloisonne ™ Rouge Flambe (ENGELHARD), 0.50% Cellini Red (ENGELHARD).
⁶Comprises 53% PPG-26-buteth-26, 37% PEG-40 hydrogenated castor oil and 10% water.
⁷Comprises 80% behentrimonium chloride and 20% isopropyl alcohol.

In a suitable container, mix the polyquatemium-37 and the Solubilisant LRI, together with approximately three-fourths of the water to produce a clear gel (mixture A). In a separate suitable container, mix the remaining ingredients, including the remaining water, heating to 70° C. while stirring, until all the ingredients are dissolved (mixture B). Cool mixture B and add slowly to mixture A while stirring. Transfer to a suitable storage container. Apply the resulting composition to clean, wet hair. Do not rinse or remove excess composition from the hair until the next washing. Style as desired.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description are, in relevant part, incorporated herein by reference; the citation of any document is not to be construed as an admission that it is. prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A leave-on hair care composition, comprising:
a) from about 0.1% to about 10% of a shine enhancing system comprising a first non-soluble particle reflecting a first color, a second non-soluble particle reflecting a second color, and a third non-soluble particle reflecting a third color;
b) from about 0.001% to about 5% of a film-forming agent; and
c) a dermatologically-acceptable carrier.

2. The composition of claim 1, wherein the film-forming agent comprises from about 0.001% to about 1% of one or more sugar amines.

3. The composition of claim 2, wherein the sugar amine is selected from the group consisting of glucosamine, N-acetyl glucosamine, mannosamine, N-acetyl mannosamine, galactosamine, N-acetyl galactosamine, and mixtures thereof.

4. The composition of claim 3, wherein the sugar amine is N-acetyl glucosamine.

5. The composition of claim 2, wherein the ratio of the percentage of shine enhancing system to the percentage of sugar amine is about 10:1.

6. The composition of claim 1, wherein the first non-soluble particle, the second non-soluble particle and the third non-soluble particle have an average size of from about 1 micron to about 150 microns.

7. The composition of claim 1, wherein the first color is in the red color family, the second color is in the yellow color family and the third color is in the green color family.

8. The composition of claim 1, wherein at least one of the first, second or third non-soluble particles is selected from the group consisting of reflective particles, gonichromatic particles, photonic crystals, luminescent pigments, fluorescent pigments, and phosphorescent pigments.

9. The composition of claim 8, wherein the goniochromatic particle is an interference pigment.

10. The composition of claim 1, wherein the first non-soluble particle, the second non-soluble particle and the third non-soluble particle are interference pigments.

11. The composition of claim 1, wherein the first non-soluble particle is a reflective particle and the second non-soluble particle is an interference pigment.

12. The composition of claim 1, further comprising a dye.

13. The composition of claim 1, further comprising a fourth non-soluble particle reflecting a fourth color.

14. The composition of claim 13, wherein the fourth color is in the blue color family.

15. The composition of claim 1, further comprising at least one additional non-soluble particle reflecting at least one additional color.

16. A method of imparting vibrancy to mammalian hair, comprising the step of applying to the hair a leave-on composition comprising:
a) from about 0.1% to about 10% of a shine enhancing system comprising a first non-soluble particle reflecting a first color, a second non-soluble particle reflecting a second color, and a third non-soluble particle reflecting a third color;
b) from about 0.001% to about 5% of a film-forming agent; and
c) a dermatologically-acceptable carrier.

17. The method of claim 16, wherein the mammalian hair is human hair.

18. The method of claim 16, wherein the mammalian hair is selected from the group consisting of dog hair, cat hair, and combinations thereof.

19. The method of claim 16, wherein the first color is in the red color family, the second color is in the yellow color family and the third color is in the green color family.

20. The method of claim 19, wherein the hair has a blonde base hair color.

21. The method of claim 19, wherein the ratio of the percentage of the first non-soluble particle to the percentage of the second non-soluble particle to the percentage of the third non-soluble particle is about 1:1:3.

22. The method of claim 16, wherein the composition further comprises a fourth non-soluble particle reflecting a fourth color.

23. The method of claim 22, wherein the fourth color is in the blue color family.

24. The method of claim 23, wherein the hair has a base hair color of brunette, black, and combinations thereof.

25. The method of claim 22, wherein the ratio of the percentage of the first non-soluble particle to the percentage of the second non-soluble particle to the percentage of the third non-soluble particle to the fourth non-soluble particle is about 2:2:1:1.

26. The method of claim 16, wherein the composition is applied to a portion of the mammalian hair.

27. The method of claim 16, further comprising the step of allowing the composition to remain on the hair for the period of time beginning after a first washing of the hair and ending at a second washing of the hair.

28. The method of claim 27, wherein the period of time comprises about twenty-four hours.

29. A kit comprising:
a) a leave-on hair care composition comprising:
i. from about 0.1% to about 10% of a shine enhancing system comprising a first non-soluble particle reflecting a first color, a second non-soluble particle reflecting a second color, and a third non-soluble particle reflecting a third color;
ii. from about 0.001% to about 5% of a film-forming agent; and
iii. a dermatologically-acceptable carrier;
b) at least one additional composition; and
c) instructions for complying with a hair treatment regimen.

30. The kit of claim 29, further comprising at least one additional component, selected from the group consisting of implements, applicators, combs, brushes, styling aids, decorative adornments, one or more orally ingestible dietary supplements, a delivery enhancement device, instructions for use of the device, and combinations thereof.

31. The kit of claim 29, wherein the additional composition is selected from the group consisting of a hair colorant, a hair dye, a shampoos, a conditioner, a styling composition, and combinations thereof.
